(19) United States
(12) Patent Application Publication
(10) Pub. No.: US 2004/0102363 A1
(43) Pub. Date: May 27, 2004

(54) Substantially pure lectin for prevention and treatment of crustaceous infected by baculovirus, a food composition containing the lectin and a method for increasing the crustaceous resistance to infections

(76) Inventor: Luis Alberto Romano, Buenos Aires (AR)

Correspondence Address: GOTTLIEB RACKMAN & REISMAN PC, 270 MADISON AVENUE, 8TH FLOOR, NEW YORK, NY, 100160601

(21) Appl. No.: 10/410,838

(22) Filed: Apr. 10, 2003

(30) Foreign Application Priority Data
Apr. 11, 2002 (AR) P020101342

(51) Int. Cl. A61K 38/17; C07K 14/435

(52) U.S. Cl. 514/8; 530/396

(57) ABSTRACT

A substantially pure lectin for prevention and treatment of crustaceous infected by baculovirus, a food composition containing the lectin and a method for increasing the crustaceous resistance to infections. The lectin is isolated from the species Macrobrachium rosenbergii, and it has the aminoacid sequence SEQ ID No. I, wherein the complete sequence or part thereof is employed for elaborating a food composition for treating the water of a prawn breeding station, in a manner to prevent and/or cure the prawn infected by the baculovirus that causes the “White spot” syndrome.

The present invention relates to a substantially pure lectin for prevention and treatment of crustaceous infected by baculovirus, a food composition containing the lectin and a method for increasing the crustaceous resistance to infections. More specifically, the invention relates to a lectin that is isolated from the species Macrobrachium rosenbergii, that has the aminoacid sequence SEQ ID No. I, and that the complete sequence of the lectin or part thereof is employed for elaborating a food composition for treating the water of a prawn breeding station, in a manner to prevent and/or cure the prawn infected by the baculovirus that causes the “White spot” syndrome.

### PRIOR ART OF THE INVENTION

Recently it has been detected the appearance of the white spot syndrome (WSS) in several prawn and scrimp breeding stations in Central America, that bars the sea crustaceous production in this region. Also some cases have been registered in the east coast of the USA and the Mexican gulf. This is specially important when considering that the prawn and shrimp breeding has been one of the economic activities with largest productive growing in the last fifteen years in Latin America and particularly in Ecuador.

The viral diseases produced by the WSSV from the 90's till the present have caused just in Ecuador damages estimated in one million dollars.

This baculovirus already had a great importance in the Asian aguiculture, however it was not until recent years that the virus was considered a dangerous agent for the occidental sea culturing. However, like other pandemic processes that begin in a concrete site to then spread all around the world, the WSSV may be considered today as a cosmopolitan disease giving more importance to the fact that it is a devastating disease in economic and ecological terms.

The white spot syndrome has been described at the beginning of the decade in Asia and other countries traditionally involved in the sea crustaceous breeding. This infection has been named differently depending on the geographical region, in Japan, for example, it is called peneidae bacillary virus (PRDV); in China the disease is attributed to the Hypodermic and Hemopoyetic Necrosis Baculovirus (HHNBV); in Thailand the agent causing the disease is called Mesodermal Ecto Baculovirus (SEMBV). Then, by means of studies employing genetic techniques it has been determined that these agents may be grouped into the WSSV complex, and that there existed a few differences between them.

From the clinical point of view, the infection with WSSV is characterized by the appearance of white spots in the inner face of the affected crustaceous cuticle. The appearance of these spots not necessarily involves the presence of other clinic symptoms, therefore the crustaceous having no other symptoms are considered in a prepatency stage without the disease being developed.

The disease generally appears as a result of a stress situation and it is manifested with lethargy, redness, anorexia, superficial swimming and dead in hours or in few days. Mortality may reach the 100% of the affected population.

The WSSV is characterized by being highly virulent and little specific as to its capacity for invading and damaging several tissues. By means of histopathology techniques evidences it has been found relating to infection and destruction of connective tissue cells, antenna gland, hematopoietic tissue, branchias, nervous tissue, ectoderm and lymphoid organs, the two last ones being the more frequently affected.

The transmission of WSSV is produced either by cohabitation with infected crustaceous, for example, and under experimental conditions with high concentrations (10⁻⁷/ml) of the virus in the water that have shown to be infecting. The virus also can be infected by ingestion and in a vertical way. This latter transmission way is a strong economic impact because the larvae died before the appearance.

One of the largest difficulties in beginning with eradication plans is that the wild species are affected and behave like natural reservoirs, for example, several grab and prawn or shrimp species living in a free environment are capable of keeping and transmitting the WSSV.

The most employed methods for diagnosing this disease have been, until recent years, those employing histopathology techniques and observation of impressions by classic microscopy. The electronic microscopy has also been employed to describe the disease. These methods, combined with the clinical symptomatology, served to describe the first cases of the WSSV disease. Bacyllar shape viral particles, with tri-laminar coverings, typical aspect of the baculovirus, have been observed by electronic microscopy, in several tissues of an infected crustaceous.

Because there are several baculovirus affecting the crustaceous and, as remarked above, just after some years of studies all the isolated strains have been grouped, taking as a starting point the WSS, the use of more specific techniques based in the DNA detection have permitted a great progress in the diagnosis of this disease. Among these techniques it may be cited the hybridation “in situ” and the polymerase chain reaction (PCR), this one being of the single or double type.

Nowadays there are few treatments for fighting against the disease, generally only the disease prevention being applied.

Prevention fundamentally consists of certain recommendations useful to reduce the risk and spreading of the disease. In regions not affected by the endemic form of the WSSV, the main prevention form is the acquisition of animals from WSSV-free factories.

It is known from experiments that a high concentration of the virus may persist in the sea water up to 120 days. However, in natural concentrations the virus do nor persist more than 5-7 days, a time enough, however, to disseminate the infection. Some of the disinfection methods that have been efficient are the use of sodium hypochlorite at a concentration of 10 ppm for 5 minutes; the iodine povidone at the same concentration and doses, or at concentrations of 15% in sodium chloride.

Other methods that have been used experimentally are the use of ultraviolet radiation, extreme pH (pH 3 for 1 hour), or the use of ozone that produces the inactivation of the virus with T.R.O. (Total Oxidizing Residue) f 0.5 μg/ml.

The disinfection after a sanitary vacuum, the acquisition of WSSV-free animals, together with a safety diet free of elements coming from prawn, shrimp or other crustaceous for avoiding, as much as possible, the natural reservoirs, are the present norms and guidelines for fighting against this disease. At present does not exist an efficient treatment for eliminating the viral infection once the same has been established in the crustaceous living zone or breeding station.

### BRIEF DESCRIPTION OF THE INVENTION

It is therefore an object of the present invention to provide a substantially pure lectin for prevention and treatment of crustaceous infected by baculovirus, characterized in that said lectin is isolated from the species Macrobrachium rosenbergii, and wherein said lectin has an aminoacid sequence with an identity that is between 70% and 99% of the aminoacid sequence SEQ ID No. I or part thereof, such as the active domains. Said crustaceous under treatment may be prawn, shrimp and crab and the baculovirus is WSSV.

It is another object of the present invention to provide a food compound for treating diseases related to the baculovirus, wherein the composition comprises a lectin isolated from the species Macrobrachium rosenbergii, and wherein said lectin has an aminoacid sequence with an identity that is between 70% and 99% of the aminoacid sequence SEQ ID No. I or part thereof.

In a preferred embodiment the composition comprises at least between 40%/w and 60%/w of the lectin; between 0.5%/w and 1%/w of proper preservatives and between 59.5%/w and 39%/w of proper food supplements.

In another inventive object a method for increasing the crustaceous resistance to baculovirus infections is provided, wherein the method comprises treating the aqueous medium of a crustaceous breeding tank with a composition comprising at least one lectin having an aminoacid sequence with an identity that is between 70% and 99% of the aminoacid sequence SEQ ID No. I or part thereof, and proper food supplements and preservatives. Wherein said composition is added to the water in a 1:1 ratio regarding the biomass.

### DETAILED DESCRIPTION OF THE INVENTION

It is well known that the lectin of the invertebrate such as the crustaceous participates in the immunity against infections, activating the profenoloxidase system.

The inventors have isolated a novel lectin corresponding to a prawn species (Macrobrachium rosenbergii) that is resistant to the infection from WSSV; and they have demonstrated that said lectin can provide immunity to animals sensitive to the WSSV. The use of said lectin permits both the prevention and the treatment of infected prawn.

The inventive lectin has shown to have specific erythro agglutinant activity like a filtrated and purified crude extract of Macrobrachium rosenbergii. This is shown in Table 1 where it can be seen that any of the three lots resulted agglutinant for the blood system ABO.

TABLE 1
Behaviour of the erythro agglutinant activity of the
isolated lectin from Macrobrachium rosenbergii

| | Crude Extract | Filtrated Extr. | Purified Extr. |
|---|---|---|---|
| Lot | ABO | ABO | ABO |
| I | Agglutination | Agglutination | Agglutination |
| II | Agglutination | Agglutination | Agglutination |
| III | Agglutination | Agglutination | Agglutination |

It was also demonstrated that the agglutinating capacity of the inventive lectin is inhibited in the presence of glucose, thus showing that the same recognizes and joins the glucose.

For evaluating the performance of the lectin purification three lots of lectin purification have been made from the hepatopancreas of Macrobrachium rosenbergii. The results may be seen in Table 2.

TABLE 2
Performance of the lectin purification of Macrobrachium
rosenbergii, expressed as total proteins

| | | Filtrated | Purified | |
|---|---|---|---|---|
| | | extract | extract | |
| | Hepatopancreas | (proteins in | (proteins in | Performance |
| Lot | weight (gr.) | gr.) | gr.) | (%) |
| I | 50 | 1.15 | 0.84 | 73 |
| II | 50 | 1.12 | 0.84 | 75 |
| III | 50 | 1.07 | 0.9 | 84 |

The performance or yielding in the recuperation of lectin was about 77% what may be considered an optimum value for this type of proteins.

The gel electrophoresis test shown that the purified extracts corresponded to the lectin and no other molecular or contaminant species have been found.

Subsequently, the purified lectin was sequenced, said sequence of aminoacids correspond to SEQ ID No. 1 disclosed below:

SEQ ID NO 1:

sypntdigdpsyphigidiksirskstarwnmqtgkvgtahisadtivaveldynsvak rlsavvsytgsssttvsydvdlnlsattglyketntilswsftsklktnsiadannvlp ewvrvgVPTQRLDA.fs.qnpkDLILTTDOSDASD.GNLELTkvss.sgdpqgsLPSQH LVEALFVCGHIWe . . . kSAGFDATFTAVVFLIks . . . pdrdPGITFNTDTFIAADSip sgsLFGLGDRFPDAN

For the purposes of the present invention the complete lectin or a lectin having between 70% and 90% homology with the SEQ ID No. 1 or part thereof, for example the domains indicated in the SEQ ID No. 1 or any other part of said aminoacid sequence with anti-WSSV activity, may be employed for the elaboration of the inventive food composition.

In the preparation of the inventive food composition food supplements and conserving agents are mixed with the lectin or part thereof. Any person skilled in the art will understand that any food supplement providing the proper quantity of proteins, lipids and carbohydrates that are necessary for a correct feeding of the prawn or other crustaceous may be employed.

For the preparation of the food composition the dry elements may be agglutinated with any agglutinating compound such as fish oil, gelatin and others.

It was shown that the incorporation of the isolated lectin from Macrobrachium rosenbergii in the food composition induced resistance of the specimen Litopenaeus vannamei against the white spot disease virus. While the control groups have shown a 100% mortality, the treated groups had a surviving of between 65.3% and 98.7% as it shown in Table 3.

TABLE 3
Experimental groups and surviving distribution

| | N° of | | Treating: gr. | |
|---|---|---|---|---|
| | Litopenaeus | | Of lectin per | |
| Experimental | vannamei | Infection | Kg. Of food | Surviving |
| groups | specimens | conditions | composition | in % |
| 1 | 100 | Infected with | 10 | 78.4 |
| | | WSSV 50 ml/l | | |
| 2 | 100 | Infected with | 10 | 98.4 |
| | | WSSV 25 ml/l | | |
| 3 | 100 | Infected with | 10 | 63 |
| | | WSSV 10 ml/l | | |
| 4 | 100 | Infected with | 10 | 98.7 |
| | | WSSV 5 ml/l | | |
| 5 | 100 | Infected with | 10 | 85.4 |
| | | WSSV 1 ml/l | | |
| 6 | 100 | Infected with | Without | 0 |
| | | WSSV 50 ml/l | treating | |
| 7 | 100 | Infected with | Without | 0 |
| | | WSSV 25 ml/l | treating | |
| 8 | 100 | Infected with | Without | 0 |
| | | WSSV 10 ml/l | treating | |
| 9 | 100 | Infected with | Without | 0 |
| | | WSSV 5 ml/l | treating | |
| 10 | 100 | Infected with | Without | 0 |
| | | WSSV 1 ml/l | treating | |

For determining the extent of infection in the animals these ones were evaluated clinically, histologically and by PCR.

The inventive composition may be administered into the water of the crustaceous breeding tank in concentrations varying on the stage of the crustaceous evolution. For example, the food composition may be added in the following ratio, one part of food per one part of biomass, in relation to the developing stage.

Given the relationship existing between the different baculovirus and considering the fact that the WSSV infects other types of crustaceous other than the prawn and shrimps, the inventive lectin and the composition containing the same may be employed in the treatment of a large variety of crustaceous, in addition to the prawns and shrimps, such as all those crustaceous affected by the virus.

### EXAMPLE 1

### Obtainment and Purification of Lectin from Macrobrachium rosenbergii

The lectin was obtained by the method disclosed by Agrawall and Goldstein. Briefly, 50 gr. Of hepatopancreas were extracted from M. rosenbergii and were deposited into an agitator containing a saline solution of 0.15 sodium chloride at 4° C., for 4 hours. Then, the tissue was homogenized under cold conditions and the saline solution was added until reaching the 1:5 weight/volume ratio. Soft agitation was applied for 24 hours at 4° C. The pH was adjusted to 4.7 and the mass was centrifuged at 3,000 rpm for 30 minutes at 4° C., the supernatant was conserved under cold conditions and the extraction process was repeated. The extractions were mixed, their agglutinant activity was determined and ammonium sulfate was added up to reaching a 18% saturation, and the pH was adjusted to 7.0. The solution was let settle for 24 hours and it was eliminated by centrifugation and ammonium sulfate was added to the supernatant up to reaching a 60% saturation, the pH was adjusted to 7.0 and it was let settle under cold conditions for 24 hours, the precipitate was separated by centrifugation and the precipitate was diluted in 100 ml-150 ml water, it was exhaustively dialyzed against water and saline solution of 1 mol of NaCl, successively. The formed precipitate was eliminated and the supernatant was clarified by filtration (0.45μ). The protein concentration and the agglutinant activity of the extract were determined.

The purification was carried out by affinity chromatography in a Sephadex G-50 matrix finely packaged into column XK-50/15. The column was washed with saline solution 1 mol of sodium chloride for 48 hours. The lectin retained in the column was removed by passing a 0.1 mol solution of D+glucose in saline medium at a low speed flow. All the process was carried out in an automatic system FPLC with monitoring of the eluates at 280 nm. The eluates with glucose were joined and dialyzed with 0.1 mol NaCl saline solution. The solution was concentrated up to levels close to 5 mg/ml. Appropriate concentrations of calcium and manganese ions were added.

The proteinaceous samples were subject to a gel electrophoresis technique. For this purpose solutions between 0.3 mg/ml and 0.6 mg/ml were separated. In all cases 0.3 μl of the patterns and the samples over Phastgeles 8-25% gradients were applied. A Fast-System equipment was used and, as revealers, the Coomasie reagents and a Silver Nitrate solution were employed.

The lectin was sequence by appropriate methods and sequencer.

### EXAMPLE 2

### Preparation of the Inventive Food Composition

1.9 gr. wheat flour, 4 gr. fish powder, 16 gr. bee pollen, 78 gr. lectin of the invention and 0.1 gr. conserving agent were mixed together. A sufficient quantity of fish oil was added as agglutinant to the mixture of the above dry ingredients.

### EXAMPLE 3

### Testing the Inventive Composition in the Treatment of WSSV-Infected Prawns

10 lots of young prawns or shrimps from Litopenaus vannameii were prepared and separated into 10 experimental models as shown in the following Table.

TABLE 4
Distribution of the experimental groups

| | N° of | | |
|---|---|---|---|
| | Litopenaeus | | Treating: lectin |
| Experimental | vannamei | Infection | gr. per food |
| groups | specimens | conditions | composition Kg. |
| 1 | 100 | Infected with WSSV | 10 |
| | | 50 ml/l | |
| 2 | 100 | Infected with WSSV | 10 |
| | | 25 ml/l | |
| 3 | 100 | Infected with WSSV | 10 |
| | | 10 ml/l | |
| 4 | 100 | Infected with WSSV | 10 |
| | | 5 ml/l | |
| 5 | 100 | Infected with WSSV | 10 |
| | | 1 ml/l | |
| 6 | 100 | Infected with WSSV | Without treating |
| | | 50 ml/l | |
| 7 | 100 | Infected with WSSV | Without treating |
| | | 25 ml/l | |
| 8 | 100 | Infected with WSSV | Without treating |
| | | 10 ml/l | |
| 9 | 100 | Infected with WSSV | Without treating |
| | | 5 ml/l | |
| 10 | 100 | Infected with WSSV | Without treating |
| | | 1 ml/l | |

The prawns or shrimps were maintained in aquariums under controlled environmental conditions. The experimental infection was made by incorporating into the water a liquid obtained by homogenization of hepatopancreas from adult animals having clinical signs of the white spot disease (WSSV). The liquid was controlled by PCR.

The tracking of the virus was made by PCR, for this purpose samples of the water of each of the aquariums, the virus concentrated liquid, the homogenized prawn hepatopancreas and prawn cuticle macerations were taken.

The samples were washed with TE 50 ml (1M), 4 ml EDTA (0.5 M), 0.2 ml NaCl (5M), 100 ml distillated water, the PH was adjusted to 9.0 with NaOH. The buffer solution was changed 4 times during 24 hours. Subsequently, the samples were maintained in a double boiler at 55° C. for 15 hours in a solution containing 10 ml buffer solution TE9, 0.10% SDS and 25 μl proteinase K. Then, the samples were transferred into a test tube and phenol-chloroform was added. It was centrifuged at 10,000 rpm for 10 minutes in aqueous phase and the material was removed. The final extraction of the aqueous phase was made after the addition of 1 vol. chloroform and centrifugation. The aqueous phase was removed from each sample and it was placed into a tube, 1 vol. sodium acetate (3M) solution was added. The samples were precipitated with 1 vol. ethanol 95% and they were conserved at −20° C. for at least 12 hours. Then, each sample was centrifuged at 10,000 rpm for 10 minutes and the supernatant was carefully removed. Each DNA sample was washed with ethanol 70% and was washed again. The ethanol was removed and the DNA was kept for some minutes at room temperature for total evaporation. Each sample was re-suspended in a solution of Tris (3 mM)—EDTA (0.2 nM), pH 7.2.

The PCR reaction was made in a thermo-cycling device with the following cycles: 94° C. for 5 minutes, 40 cycles at 94° C. for 1 minute, 55° C. for 1 minute and 72° C. for 2 minutes, finally 72° C. for 5 minutes, the reaction was stopped. The PCR technique was made by employing a kit commercially available from the Cipres Laboratory, Belgium.

The reaction products from the PCR were characterized by agarose gel electrophoresis.

Having specially described and determined the nature of the present invention and the way it may be reduced to practice the following is claimed for our property and exclusive rights:

1. A substantially pure lectin for prevention and treatment of crustaceous infected by baculovirus, characterized in that said lectin is isolated from the species Macrobrachium rosenbergii, and wherein said lectin has an aminoacid sequence with an identity that is between 70% and 99% of the aminoacid sequence SEQ ID No. I or part thereof.

2. The lectin of claim 1, characterized in that the crustaceous are selected from the group comprising prawn, shrimp and crab.

3. The lectin of claim 1, characterized in that the baculovirus is WSSV.

4. A food compound for treating diseases related to the baculovirus, comprising the lectin of claim 1, characterized by at least:
between 40%/w and 60%/w of the lectin of claim 1;
between 0.5%/w and 1%/w of proper preservatives; and
between 59.5%/w and 39%/w of proper food supplements.

5. The composition of claim 4, characterized in that the food supplements comprise:
between 5%/w and 10%/w of proteins;
between 4%/w and 5%/w of lipids;
between 10%/w and 15%/w of carbohydrates.

6. The composition of claim 5, characterized in that the food complements comprise:
between 1.9%/w and 2%/w of wheat flour;
between 3.9%/w and 4%/w of fish powder;
between 15.9%/w and 16%/w of bee pollen.

7. The composition of claim 4, characterized in that the food preservatives comprise:
between 0.5%/w and 1%/w of a mixture of BHT and BHA.

8. A method for increasing the crustaceous resistance to baculovirus infections by using the composition of claim 4, characterized by treating the aqueous medium of a crustaceous breeding tank with a composition comprising at least one lectin having an aminoacid sequence with an identity that is between 70% and 99% of the aminoacid sequence SEQ ID No. I or part thereof, and proper food supplements and preservatives.

9. The method of claim 8, characterized in that the baculovirus is WSSV.

10. The method of claim 8, characterized in that the crustaceous are selected from the group comprising prawn, shrimp and crab.

11. The method of claim 8, characterized in that the composition is added to the aqueous medium in a ratio 1:1 regarding the biomass.
